(19) United States
(12) Patent Application Publication
(10) Pub. No.: US 2010/0100028 A1
(43) Pub. Date: Apr. 22, 2010

(54) APPLICATORS HAVING IMPROVED LIGHT SCATTERING PROPERTIES

(76) Inventors: Steven Ray Gilbert, Fairfield, OH (US); Susan Louise Joa, Liberty Township, OH (US); Jeffrey Scott Hudson, Cincinnati, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/254,858

(22) Filed: Oct. 21, 2008

(51) Int. Cl. A61F 13/26

(52) U.S. Cl. 604/11

(57) ABSTRACT

A biodegradable applicator for a feminine hygiene product. The applicator has an outer member having an insertion end, a withdrawal end and a viewing surface. The viewing surface can have a delta hue greater than about three degrees at a reflectance angle less than about 25 degrees, a flop index greater than about 5, and/or a luminosity greater than about 1600.

### FIELD OF THE INVENTION

The present application relates generally to applicators and more particularly relates to applicators having improved light scattering properties.

### BACKGROUND OF THE INVENTION

Feminine hygiene products such as tampons are commonly used by female consumers. Such feminine products can be inserted into the user's vagina digitally, such as, e.g., by using a finger, or can be inserted into the vagina by using an applicator. Applicators typically comprise an outer tubular member and a plunger for insertion of the tampon, and can be made from suitable materials such as, e.g., paper and/or plastic materials that retain their form during use and are shelf-stable under ambient conditions.

Although paper applicators are considered environmentally friendly in that they can readily disintegrate in a sewage system and/or can be disposed of through aerobic, anaerobic, and natural degradation processes, some consumers may prefer plastic applicators due to a perception of smooth and comfortable insertion. Consumers may also be attracted to the smooth pearlescent appearance of the plastic material, e.g., a pearly essence or gloss, and/or may associate this smooth appearance with a comfortable insertion experience. The same appearance, however, generally cannot be duplicated in products and/or packaging made from paperboard, cardboard, or other types of biodegradable and/or flushable materials.

As such, there is a need for an improved applicator, associated packaging, and other types of products or packaging that may be biodegradable and/or flushable while at the same time having the same or a similar visual appearance as plastic applicators, products, and packaging.

### SUMMARY OF THE INVENTION

A biodegradable applicator for a feminine hygiene product is provided. The applicator can have an outer member having an insertion end, a withdrawal end and a viewing surface. In certain embodiments, the viewing surface can have a delta hue greater than about three degrees at a reflectance angle less than about 25 degrees. In addition, or alternatively, the viewing surface can have a flop index greater than about 5 and/or a luminosity greater than about 1600.

These and other features of the present application will become apparent to one of ordinary skill in the art upon review of the following detailed description when taken in conjunction with the several drawings and the appended claims.

### BRIEF DESCRIPTION OF THE DRAWINGS

FIG. 1 is a perspective view of a tampon within an applicator.

FIG. 2 is a perspective view of a tampon wrapper.

FIG. 3 is a perspective view of a tampon package and a tampon package insert.

FIG. 4 shows a side cross-sectional view of a tampon applicator.

FIG. 5 shows an alternative embodiment of a tampon applicator.

FIG. 6 is a graph showing the Flop Index of various samples.

### DETAILED DESCRIPTION OF THE INVENTION

The present invention relates to applicators, such as tampon applicators, and other packaging, that have improved light scattering properties. Such applicators and packaging can provide the benefits of cardboard or paperboard applicators while having an appearance more closely approaching that of plastic applicators and packaging. For example, in certain embodiments, the applicator can be formed of cardboard or paperboard and can have light scattering properties approximating the light scattering properties of an applicator formed of plastic, which can provide a user with the perception of smooth, comfortable insertion and improved visual appearance, combined with the biodegradability and cost savings that may be associated with a paper applicator.

Section A below describes terms for assisting the reader in understanding the features of the application, but does not introduce limitations into the terms that may be inconsistent with the context in which they are used in the specification herein. Section B is the detailed description of the drawings illustrating the applicator and the other packaging elements in accordance with the embodiments of this application. Section C describes the various light scattering applications described herein.

A. Terms

As used herein, the term “feminine hygiene article” refers to articles that typically can be intended for feminine use, such as, e.g., sanitary napkins, liners, tampons, interlabial articles, incontinence articles, and pessaries.

As used herein, the term “tampon” refers to any type of absorbent structure that can be inserted into the vaginal canal or other body cavity, such as, e.g., for the absorption of fluid, to aid in wound healing, and/or for the delivery of materials, such as moisture or active materials such as medicaments.

As used herein, the term “pessary” refers to any type of substantially non-absorbent structure for the purpose of reducing urine leakage and/or supporting a uterus and/or bladder. Such pessaries can have any variety of shapes and sizes including cylinder, ovate, spherical, tubular, annual rings, “U” shaped, cup shaped, rings, cubes or donut shaped, and can function in any suitable manner, such as, e.g., by direct application of support, lever force, expansion of the device by selection of material, and/or by inflation of the device.

As used herein, the term “vaginal canal” refers to the internal genitalia of the human female in the pudendal region of the body. The terms “vaginal canal” or “within the vagina” as used herein are intended to refer to the space located between the introitus of the vagina and the cervix.

As used herein, “applicator” refers to a device or implement that facilitates the insertion of a feminine hygiene product, such as, e.g., a tampon or pessary, into an external orifice of a mammal. Exemplary applicators include telescoping, tube and plunger, and compact applicators.

The term “flushable” as used herein refers to materials which are capable of dissolving, dispersing, disintegrating, and/or decomposing in a toilet to provide clearance when flushed down the toilet without clogging the toilet or any other sewage drainage pipe.

The term “water-dispersible” as used herein refers to materials that readily break apart in unrecognizable pieces upon contact with water as a result of dissolution, solubilization, dissipation, agitation, softening, or any other chemical or mechanical dispersion means.

The term “biodegradable” as used herein refers to materials that when disposed of after use will physically and biologically decompose using known degradation procedures including aerobic, anaerobic, and microbial digestion processes. In addition, the biodegradable materials described herein include those degradable water-insoluble materials that will physically and biologically decompose after disposal in a sewage system. In certain embodiments, the biodegradable materials can degrade in a suitable amount of time such as, e.g., less than about a year, less than about a month, less than about a week, and/or less than about a day.

B. General Description of a Tampon Applicator and Packaging

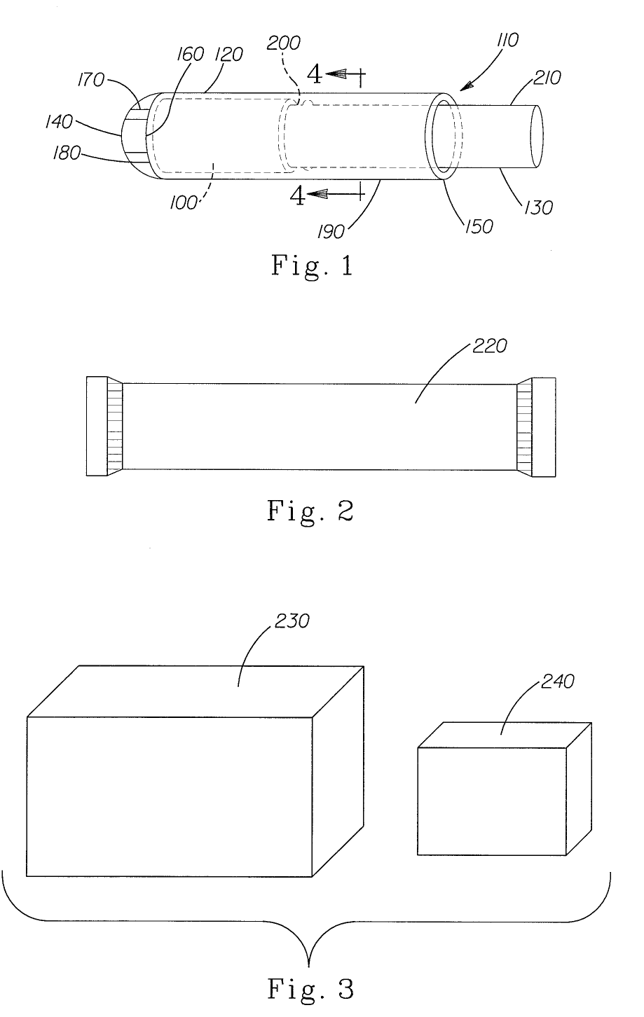

Referring now to the drawings in which like numerals refer to like elements throughout the several views, FIG. 1 shows a tampon 100 positioned within a tampon applicator 110. Although a tampon 100 and an applicator 110 are described herein, any type of feminine hygiene or other type of consumer product may be used. The tampon applicator 110 may be used with any type of tampon 100. The tampon applicator 110 may be made from rolled cardboard or other types of flushable, degradable materials. Any suitable size or shaped applicator may be used.

As shown in FIG. 1, the tampon applicator 110 may include an outer member 120 and an inner member 130. The outer member 120 may include an insertion end 140 and a second end 150 opposed to the insertion end 140. In certain embodiments, a preformed hinge or groove 160 may extend around the periphery of the outer member 120 near the insertion end 140. In addition, or alternatively, the outer member 120 also may have a domed shaped end having a number of radial slits 170 extending from a central aperture, such as, e.g., to the groove 160. In certain embodiments, the portion of the outer member 120 adjacent to the insertion end 140 also may have an openable end and may include a number of petals 180. The insertion end 140 may be opened in whole or in part. The outer member 120 also may have a grip region 190 positioned about the second end 150.

The inner member 130 may be dimensioned to slide within the outer member 120, such as, e.g., with minimal clearance therebetween. The inner member 130 may have a first end 200 and a second end 210. The first end 200 provides the necessary structure to expel the tampon 100 from the outer member 120. The inner member 130 may be solid or hollow in whole or in part. Other types of tampon applicators 110 may be used herein.

FIG. 2 shows a wrapper 220 for enclosing the tampon 100 and the applicator 110. The wrapper 220 may made out of paper or similar materials although any type of suitable material may be used herein. The tampon 100 and the applicator 110 generally may be individually wrapped within the wrapper 220 although other configurations may be used herein. The wrapper 220 may have any desired size or shape. The wrapper 220 may have graphics and/or other types of communication positioned thereon.

FIG. 3 shows a package 230 for enclosing the tampon 100 and the applicator 110. The package 230 may be a conventional box of any shape or size and may be made out of paperboard, cardboard, or any conventional type of material. In certain embodiments, the package 230 can be a bag or any other suitable package 230. The package 230 may be constructed and enclosed by any known means. Any number of tampons 100 and applicators 110 may be positioned within the package 230. The package 230 may have graphics and/or other types of communication thereon.

A packaging insert 240 also may be positioned within the package 230. The packing insert 240 may be made out of paper or any other type of material. The insert 240 may include one or more communications to the consumer. These communications may include instructions for use, instructions for disposal, safety precautions or warnings, product information, advertising, coupons, or any other desired type of communication. Any number of inserts 240 may be used.

C. Light Scattering Applications

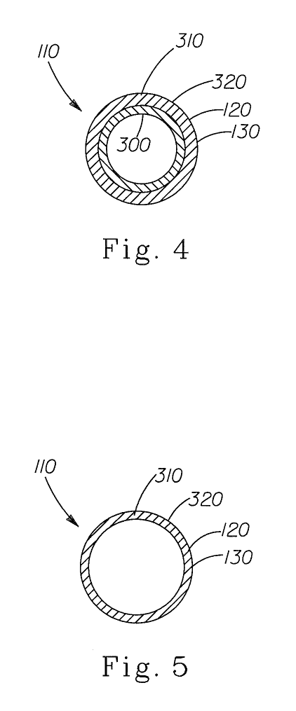

A number of different products with light scattering properties may be described herein. FIG. 4 shows a cross-section of the outer member 120 and/or the inner member 130 of the tampon applicator 110. The tampon applicator 110 has a viewing surface 320. As shown in FIG. 4, the applicator 110 can have one or more inner layers 300. The applicator 110 also may have one or more outer layers 310, and one or more of outer layers 310 can form the exterior surface 320. In certain embodiments, the exterior surface 320 can form the viewing surface 320. As shown in FIG. 4, the inner layers 300 can be a first material and the outer layers 310 can be a second material, such as, e.g., where the first material and the second material are different. For example, one or more of inner layers 300 can be a first material and can form the interior surface 330 of the applicator 110 and one or more of outer layers 310 can be a second material and can form the exterior surface 320 of the applicator 110. In addition, the inner layers 300 and the outer layers 310 may be made from a biodegradable and/or flushable material.

FIG. 5 shows an alternative embodiment of the outer member 120 and/or the inner member 130 of the applicator 110. The tampon applicator 110 has a viewing surface 320. As shown in FIG. 5, in certain embodiments, the exterior surface 320 and the interior surface 330 can be formed from the same material. In addition, in certain embodiments, the entire outer member 120 can be made of a single material.

The outer layers and inner layers can be constructed from any suitable biodegradable, flushable, and/or water dispersible materials. Suitable materials include, for example, paper, paperboard, cardboard, cellulose, cellulose acetate, cellophane, polylactic acid, polyvinyl acetate, polyvinyl alcohols, and similar types of biodegradable and/or flushable materials. In certain embodiments, the inner material and outer material can be different. For example, in certain embodiments, the inner layers can be constructed from paper, paperboard, and cardboard, and the outer layer can be constructed from cellulose, cellulose acetate, or cellophane. In addition, one or more layers of the applicator can include additives such as mica or other types of particulates.

In certain embodiments, the insertion member can be in the form of a spirally wound, convolutely wound or longitudinally seamed hollow tube. The insertion member can have one or more walls of any suitable thickness. In certain embodiments, the one or more walls can have a predetermined thickness of from about 0.1 millimeters to about 0.7 millimeter. The wall can be constructed from a single material, such as, e.g., a single ply or layer of material, or one or more materials forming one or more plies, or can be formed from two or more plies or layers, such as, e.g., two or more plies that are bonded together, such as, e.g., to form a laminate. When two or more plies are utilized, some or all of the plies can be spirally wound, convolutely wound or longitudinally seamed to form an elongated cylinder. For example, in certain embodiments the wall can be constructed using a smooth thin ply of material on the outside or exterior surface that surrounds a coarser and possibly thicker ply. In certain embodiments, the wall can be constructed using a ply of material on the outside or exterior surface that can be a film, such as, e.g., cellulose, cellulose acetate, or cellophane, and that can surround one or more plies of paperboard, paper, cardboard, or the like. In embodiments where the wall contains at least three plies, the middle ply can be the thicker ply and the interior and exterior plies can be smooth and/or slippery to facilitate expulsion of the tampon and to facilitate insertion of the insertion member. The wall can contain one to four plies, although more plies can be utilized if desired.

The plies can be held together in any suitable manner, such as, e.g., by one or more adhesives, such as glue, by heat, by pressure, by ultrasonics, by application of one or more solvents, or by any other suitable manner for holding the plies together. The adhesive can be either water-soluble or water-insoluble. In certain embodiments, a water-soluble adhesive can be used such that the wall will quickly break apart when it is immersed in water, such as, e.g., by flushing the insertion member down a toilet.

Alternatively, the material can be overlapped into a tubular configuration, such as, for example, by spirally or convolutely winding the insertion member into a cylindrical tube. In the case of other tube construction methods such as fiber molding, no seams may be present and the corrugations could optionally be formed as part of the tube molding or forming process.

In certain embodiments, the applicator can include a gripping portion that can have a plurality of gripping formations, such as, e.g., projections, rings, ridges, ribs, embossments, depressions, grooves, and/or other gripping structures. In addition, or alternatively, the applicator can include a gripping portion that can include a material that can provide a frictional resistance for the user's fingers during the insertion of the tampon applicator into the body. Suitable materials that can provide friction include, for example, abrasive materials, high wet coefficient of friction materials, pressure sensitive adhesives, or any combinations thereof.

The tampon and applicator can be packaged in a wrapper, such as, e.g., individually packaged in a wrapper. In certain embodiments, the wrapper can be biodegradable and/or flushable, such as, e.g., a wrapper made of paper. The tampon and/or applicator may or may not be visible through the wrapper. In certain embodiments, the applicator can be individually packaged in a wrapper and a plurality of wrapped applicators can be packaged in a container such as, e.g., a bag, a box, and/or a carton. The wrapped tampons may or may not be visible to the user through a window in the package.

The package can be made of any suitable material. In certain embodiments, the package can be biodegradable and/or flexible. For example, the package can comprise one or more materials, such as, for example, cardboard, paperboard, cartonboard, chipboard, plywood, paper, card stock, fabric, natural or synthetic fibers, wood, mixtures or combinations thereof, or any other suitable material.

Although these other elements need not be biodegradable and/or flushable, the wrapper, the package, and/or the packaging insert could each have the light scattering properties as described herein. In addition, in certain embodiments, the various elements may combine colors or the colors and/or communications so as to provide a common theme or common trade dress. The combination of these elements thus may highlight a key message or design feature.

Although the present application has been described in terms of applicators and associated packaging, the techniques described herein are applicable to any type of consumer goods or packaging. Other examples include, but are not limited to, food products, pet products, personal care products, cleaning products, paper products including paper towels, facial tissue and bath tissue, automotive products, baby care products, feminine hygiene products, health care products, and combinations of these products.

The present invention is further illustrated by the following examples, which should not be construed as limiting in any way.

### EXAMPLES

### Example 1

### Light Scattering Properties

This example demonstrates tampon applicator materials having improved light scattering properties.

### Materials and Methods

Approximately one inch square samples having cellulose diacetate film (Clarifoil, Engelwood Cliffs, N.J.) over a paper layer were prepared as listed in Table 1.

TABLE 1
Test Materials

| Sample | Film | Paper |
|---|---|---|
| Silver Pearl | Silver with mica | White |
| Emboss over Black | Green embossed | Black |
| Blue Pearl | Blue | White |
| Metallic Pink | Pink | Silver |
| | | metallic |
| Unemboss over Black | Green | Black |
| Emboss over White | Green embossed | White |
| Unemboss over White | Green | White |

The light scattering properties of applicators described in Table 1 were evaluated against the light scattering properties of the known tampon plastic applicators listed in Table 2 and the known flushable cardboard applicators listed in Table 3.

TABLE 2
Known Plastic Applicators

| Name | Material | Color |
|---|---|---|
| Tampax ® Pearl White | Injection molded plastic | White |
| Tampax ® Pearl Blue | Injection molded plastic | Blue |
| Playtex ® Sport ™ | Injection molded plastic | Green |

TABLE 3
Known Flushable Applicators

| Name | Material | Color |
|---|---|---|
| Tampax ® Fresh | Flushable cardboard | Light Blue |
| Tampax ® Satin | Flushable cardboard | White |
| Playtex ® Beyond ® | Flushable cardboard | Pink |

Flop index, delta hue, and total luminosity for each sample was collected by means of a goniospectrophotometer model GSP-1B and a goniospectrophotometer color measurement system model GCMS-3B (Murakami Color Research Laboratory, Tokyo, Japan). Measurements were obtained using ASTM standard E 2194-03. Color was measured by determining the spatial distribution of reflected (or transmitted) light at selected angles of illumination and detection. The angle of incident was fixed at 90° with respect to the surface normal and the angle of observation was varied from this observation angle to 125 to −35° with respect to the surface normal in steps of 5°.

### Results

1. Flop Index

The Flop Index is a measurement of the change in reflection of a metallic color as it is rotated through a range of viewing angles. A Flop Index of zero may indicate a solid color while a very high Flop Index of 15-17 may indicate a metallic or pearlescent color. As such, the Flop Index is a measurement of light scattering behavior of materials that separates monochromatic materials from those with more dispersive properties. The Flop Index was determined as follows:

$$Flop Index = \frac{2.69 {( {L}_{15 °}^{*} - {L}_{110 °}^{*} )}^{1.11}}{{( {L}_{45 °}^{*} )}^{0.86}}$$

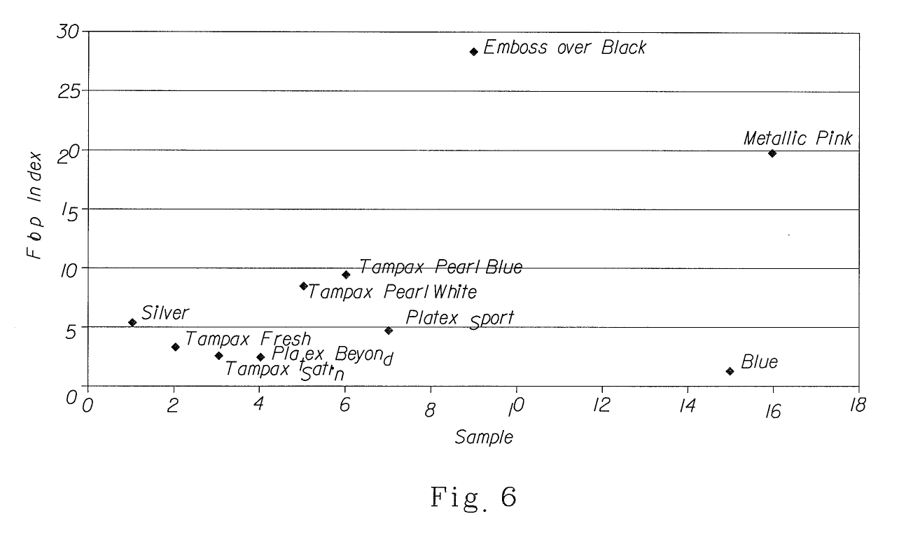

The Flop Index for each of the samples described in Tables 1-3 is shown in FIG. 6. As shown in FIG. 6, the known cardboard applicators from Table 3 have a Flop Index of about four or less, while the known plastic applicators from Table 2 have a Flop Index of about five or above. Of the test materials, Silver Pearl, Emboss over Black, Unemboss over Black, and Metallic Pink had a Flop Index of about five or above, while Emboss over White, Unemboss over White, and Blue Pearl had a Flop Index of about four or below.

2. Delta Hue

Delta hue identifies the level of color change from a given reference point with a change in angle. Hue is a measure of the color of an object and exists on a spectrum from 0 to 360 degrees, where 0 and 360 degrees is the equivalent color. Delta hue is calculated by taking the absolute value of the difference between the hue at the angle of interest minus the hue at the face angle, 45 degrees (ASTM). Because of the circular scale, the smallest arc angle distance is reported as delta hue. The degree (to the nearest 5 degree) where delta hue is greater than about three degrees is shown in Table 4.

TABLE 4
Delta Hue > About 3

| | Angle at which |
|---|---|
| | Delta Hue > |
| | About 3 Degrees |
| Tampax ® Fresh | 35° |
| Tampax ® Satin | 25° |
| Playtex ® Beyond ® | 30° |
| Tampax ® Pearl White | 5° |
| Tampax ® Pearl Blue | 10° |
| Playtex ® Sport ™ | 45° |
| Silver Pearlescent | 10° |
| Emboss Over Black | 5° |
| Blue Pearl Over White | 15° |
| Metallic Pink | 15° |
| Unemboss Over Black | 35° |
| Emboss Over White | 15° |
| Unemboss Over White | 10° |

As shown in Table 4, the known cardboard applicators exhibit a delta hue greater than about 3 degrees in the about 25° to about 35° range while the Tampax® Pearl applicators have a delta hue greater than about 3 degrees in the about 5° to about 10° range. The Silver Pearl, Emboss Over Black, Blue Pearl, and Metallic Pink test materials also have a delta hue greater than about 3 degrees in the about 5° to about 15° range in a manner similar to the known plastic applicators.

The maximum delta hue of the test materials is also greater at reflectance angles less than the face angle, i.e., below about 45 degrees, similar to the known plastic applicators. This data is represented in Table 5, which shows the maximum hue change below 45°.

TABLE 5
Maximum Hue Change

| | Maximum Hue Change |
|---|---|
| | below 45° (Face Angle) |
| Tampax ® Fresh | 12 |
| Tampax ® Satin | 52 |
| Playtex ® Beyond ® | 30 |
| Tampax ® Pearl White | 137 |
| Tampax ® Pearl Blue | 134 |
| Playtex ® Sport ™ | 24 |
| Silver Pearlescent | 78 |
| Emboss Over Black | 74 |
| Blue Pearl Over White | 196 |
| Metallic Pink | 24 |
| Unemboss Over Black | 73 |
| Emboss Over White | 17 |
| Unemboss Over White | 28 |

As shown in Table 5, the maximum hue change under 45° for the known cardboard applicators is from about 10 to about 50 degrees while the maximum hue change under 45° for the Tampax Pearl applicators is more than about 130 degrees. The maximum hue change for the Silver Pearl, Emboss Over Black, and Blue Pearl test materials ranged from about 75 to about 200 degrees. As such, several of the test materials exhibited a maximum hue change closer to the maximum hue change of the known plastic applicators as opposed to the known cardboard applicators.

3. Total Luminosity

The intensity of the light scattered also was determined using total luminosity. The total luminosity is the sum of the L* values at every 5 degree increment from 40 degrees to −35 degrees. The total luminosity below 45 degrees is shown in Table 6.

TABLE 6
Total Luminosity

| | Total Luminosity Below 45° |
|---|---|
| Tampax ® Fresh | 1530 |
| Tampax ® Satin | 1611 |
| Playtex ® Beyond ® | 1566 |
| Tampax ® Pearl White | 1931 |
| Tampax ® Pearl Blue | 1889 |
| Playtex ® Sport ™ | 1845 |
| Silver Pearlescent | 1904 |
| Emboss Over Black | 1389 |
| Blue Pearl Over White | 1767 |
| Metallic Pink | 2021 |
| Unemboss Over Black | 1254 |
| Emboss Over White | 1825 |
| Unemboss Over White | 1804 |

As is shown in Table 6, the known cardboard applicators have a total luminosity from about 1500 to about 1600, while the known plastic applicators exhibited a total luminosity of about 1800 or more. The Silver Pearl, Blue Pearl, and Metallic Pink test materials had a total luminosity greater than about 1700 or more.

In view of the above, this example demonstrates biodegradable and flushable tampon applicator materials having improved light scattering properties more closely approximating the light scattering properties of known plastic applicators compared to known cardboard applicators.

The dimensions and values disclosed herein are not to be understood as being strictly limited to the exact numerical values recited. Instead, unless otherwise specified, each dimension is intended to be the recited value and a functionally equivalent range surrounding that value. For example, the dimension disclosed as “40 mm” is intended to mean “about 40 mm.”

All documents cited in the Detailed Description of the Invention are, in relevant part, incorporated herein by reference. The citation of any document is not to be construed that it is an admission that it is prior art with respect to the present invention. To the extent that any meaning or definition of a term in this document conflicts with any meaning or definition of the same term in a document incorporated by reference or otherwise related, the meaning or definition assigned to this term in the document shall govern.

Although particular embodiments of the present invention have been illustrated and described, it would be obvious to one skilled in the art that various other changes and modifications may be made herein without departing from the general spirit and scope of the invention. It is therefore intended to cover in the independent claims all such changes and modifications that are within the scope of the invention and equivalents thereof.

We claim:

1. A biodegradable applicator for a feminine hygiene product, the applicator comprising an outer member having an insertion end, a withdrawal end and a viewing surface, the viewing surface having a delta hue greater than about three degrees at a reflectance angle less than about 25 degrees.

2. The applicator of claim 1, wherein the viewing surface has a delta hue greater than about three degrees at a reflectance angle between about 5 degrees to about 25 degrees.

3. The applicator of claim 1, wherein the viewing surface has a delta hue greater than about three degrees at a reflectance angle between about 5 degrees to about 15 degrees.

4. The applicator of claim 1, wherein the applicator comprises a first layer that is cardboard, paper, and/or paperboard.

5. The applicator of claim 4, wherein the applicator comprises a second layer that is cellulose acetate, cellulose diacetate, cellulose, cellophane, polylactic acid, polyvinyl acetate, and/or polyvinyl alcohols.

6. The applicator of claim 1, wherein the viewing surface has a flop index greater than about 5.

7. The applicator of claim 1, wherein the viewing surface has a luminosity greater than about 1600.

8. The applicator of claim 1, wherein the viewing surface has a maximum hue change below 45° of more than about 50 degrees.

9. The applicator of claim 7, wherein the maximum hue change is more than about 75 degrees.

10. The applicator of claim 7, wherein the maximum hue change is from about 60 to about 260 degrees.

11. A biodegradable applicator for a feminine hygiene product, the applicator comprising an outer member having an insertion end, a withdrawal end and a viewing surface, the viewing surface having a flop index greater than about 5.

12. The applicator of claim 11, wherein the flop index is greater than about 7.

13. The applicator of claim 11, wherein the flop index is greater than about 10.

14. The applicator of claim 11, wherein the applicator comprises a first layer that is cardboard, paper, and/or paperboard.

15. The applicator of claim 14, wherein the applicator comprises a second layer that is cellulose acetate, cellulose diacetate, cellulose, cellophane, polylactic acid, polyvinyl acetate, and/or polyvinyl alcohols.

16. The applicator of claim 11, wherein the viewing surface has a luminosity greater than about 1600.

17. A biodegradable applicator for a feminine hygiene product, the applicator comprising an outer member having an insertion end, a withdrawal end and a viewing surface, the viewing surface having a luminosity greater than about 1600.

18. The applicator of claim 16, wherein the luminosity is greater than about 1700.

19. The applicator of claim 16, wherein the applicator comprises a first layer that is cardboard, paper, and/or paperboard.

20. The applicator of claim 16, wherein the applicator comprises a second layer that is cellulose acetate, cellulose diacetate, cellulose, cellophane, polylactic acid, polyvinyl acetate, and/or polyvinyl alcohols.
